(19) United States
(12) Patent Application Publication
(10) Pub. No.: US 2025/0213283 A1
(43) Pub. Date: Jul. 3, 2025

(54) Low Profile Fixation Screws

(71) Applicant: KI Orthopedics, Inc., Woodland Hills, CA (US)

(72) Inventors: Randall Allard, Golden, CO (US); Todd Martens, Woodland Hills, CA (US)

(21) Appl. No.: 19/005,304

(22) Filed: Dec. 30, 2024

Related U.S. Application Data
(60) Provisional application No. 63/615,982, filed on Dec. 29, 2023.

(51) Int. Cl. A61B 17/86; A61F 2/30; A61F 2/42

(52) U.S. Cl. CPC A61B 17/863; A61F 2/30771; A61F 2/4225; A61F 2002/30851; A61F 2002/30866

(57) ABSTRACT

The invention relates to methods, devices, and systems for an improved bone fixation screws used as fastening devices to stabilize bone to its adjacent substrate. More particularly, the invention relates to methods, devices and systems for an improved, low profile, multi-threaded screw with mating features adapted to allow other multi-threaded screws in close proximity.

### CROSS-REFERENCE TO RELATED APPLICATIONS

This application claims the benefit of U.S. Provisional Application No. 63/615,982 entitled “Low Profile Fixation Screws” filed on Dec. 29, 2023, the disclosure of which are incorporated by reference herein in its entirety.

### TECHNICAL FIELD

The invention relates to methods, devices, and systems for an improved bone screw. More particularly, the invention relates to methods, devices and systems for an improved, low profile, multi-threaded screw with mating features adapted to allow other multi-threaded screws in close proximity.

### BACKGROUND OF THE INVENTION

Medical screws are commonly used for a variety of surgical procedures. Medical screws may be used as suture anchors to attach a suture to a bone so that the suture may be used to hold soft tissue, such as torn tendons or ligaments, adjacent to the bone. Medical screws may also be used as fastening devices to attach prosthetics such as fixation plates to bone or to join portions of a fractured bone to aid the healing process.

U.S. Pat. No. 5,743,914 (Skiba) teaches a bone screw having a head for receiving a screw driving device, a shaft having a first series of helical threads having a first diameter and a first pitch, and second series of helical threads interleaved with said first series of helical threads and having a second diameter and a second pitch, wherein the second diameter is substantially different than the first diameter and at least one of said first and second pitches changes along the length of the shaft. The helical threads are only located at the distal end portion of the screw and has different pitches along the portion of the length.

U.S. Pat. No. 5,087,201 (Mondani et al.) discloses a self-threading pin which is screw-threaded into the maxilla bone for implantation of a dental prosthetic. The pin has a screw-threaded shank portion, a driving head at one end of the shank portion and a drill bit at the other end of the shank portion. The screw-threaded shank portion of the pin has two intercalated screw threads of different heights. The helical threads are only located at the distal end portion of the screw and does not provide mating features to allow other screws to be in close proximity.

U.S. Pat. No. 11,191,576 (Champagne et al.) discloses a fixation screw with an intended to fixate intraarticular and extra-articular fractures and non-unions of small bones and bone fragments. The fixation screw also is longer than commercially available screws because it is intended to fill the intramedullary canal in substantially all its length. The helical threads are only located at the distal end portion of the screw, the shaft contains a stepped diameter or a stepped diameter transition highlighting a narrowed waisted or central area, and/or does not provide mating features to allow other screws to be in close proximity.

The bone fixation screws disclosed in the prior art generally utilize standard helical thread configurations that do not extend along the entirety of length, do not contain a low-profile first ends, and/or do not contain mating features to allow other implanted screws in close proximity.

### BRIEF DESCRIPTION OF THE SEVERAL VIEWS OF THE DRAWINGS

FIG. 1A-1J depicts several plan views and a cross-section of a first embodiment fixation screw;

FIGS. 1K-1N depicts several magnified plan views of the first embodiment fixation screw;

FIGS. 2A-2J depicts several plan views and a cross-section of a second embodiment fixation screw;

FIGS. 2K-2M depicts several magnified plan views of the second embodiment fixation screw;

FIGS. 2N-2P depicts isometric magnified views of the second embodiment fixation screw cross-sectional shape of the threads;

FIGS. 3A-3E depicts side view of one embodiment of shank body blank profiles and the corresponding fixation screws;

FIGS. 4A-4B depict flowcharts with different methods of stabilizing the capital fragment for a bunion deformity procedure; and

FIGS. 5A-5G depicts various plan views of one embodiment of a method of stabilizing the capital fragment for a bunion deformity procedure.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Although the invention is often referred to herein as a bunion fixation screw, it is understood that such description is not limiting, such that the technology in this invention may be applied in numerous other applications, including but not limited to arthrodesis of small joints, all types of bunionectomies and osteotomies, including scaphoid and other carpals bones, metacarpals, tarsals, metatarsals, patella, ulnar, styloid, capitellum, radial head and radial styloid, vertebrae (e.g., cervical, thoracic and lumbar bones), skull (e.g., occiput), and/or any other bone in adults, adolescents and/or children. In general, the order of the steps of disclosed processes may be altered within the scope of the invention.

The fixation screws disclosed herein may also be used in different surgical techniques for correcting bunion deformities. These different surgical techniques may include open, standard surgery and/or minimally invasive surgical techniques. More specifically, fixation screws may be used in the correction of bunions via the standard surgical or MIS approach that includes bunion osteotomies, bunion arthrodesis, bunion exostectomies and/or any combination thereof.

Furthermore, the fixation screws disclosed herein may also comprise compression fixation screws or non-compressive fixation screws. The compression fixation screws enable the screws to be embedded below the bone surface and generate a compression force between the desired bone portions that the fixation screw is holding together. The tightening leads to the desired bone portions to move relative to each other until the bone portions are compressed together (e.g., variable thread pitch fixation screws). The non-compression fixation screws enable the screws to hold bone portions together without or with minimal compression (e.g., uniform thread pitch fixation screws).

The invention disclosed herein intends to overcome the shortcomings of the prior art. The invention solves the problems associated with repairing a bone or metatarsal fracture, osteotomy, dislocation and/or stabilization by providing one or more bone fixation screws that are unique combinations of a nail and a screw, resulting in distinct advantages including (1) can be inserted into the bone without damaging the cortical shell or breaking of the intramedullary trabeculae; (2) a headless, low-profile design which can be seated below the skin surface to avoid the irritation that can result from an extending proud portion extending substantially away from the skin; (3) a cannulated lumen and its corresponding surgical technique designed to minimize soft tissue, cartilage and vascular damage upon insertion; (4) one or more mating scallops to facilitate close proximity stabilization; (5) tapered tip to allow centralized positioning; (6) helix cutting edges for self-tapping and/or self-threading capabilities; and/or (7) facilitation of early, active mobilization post-operative protocols for accelerated healing and earlier return to work.

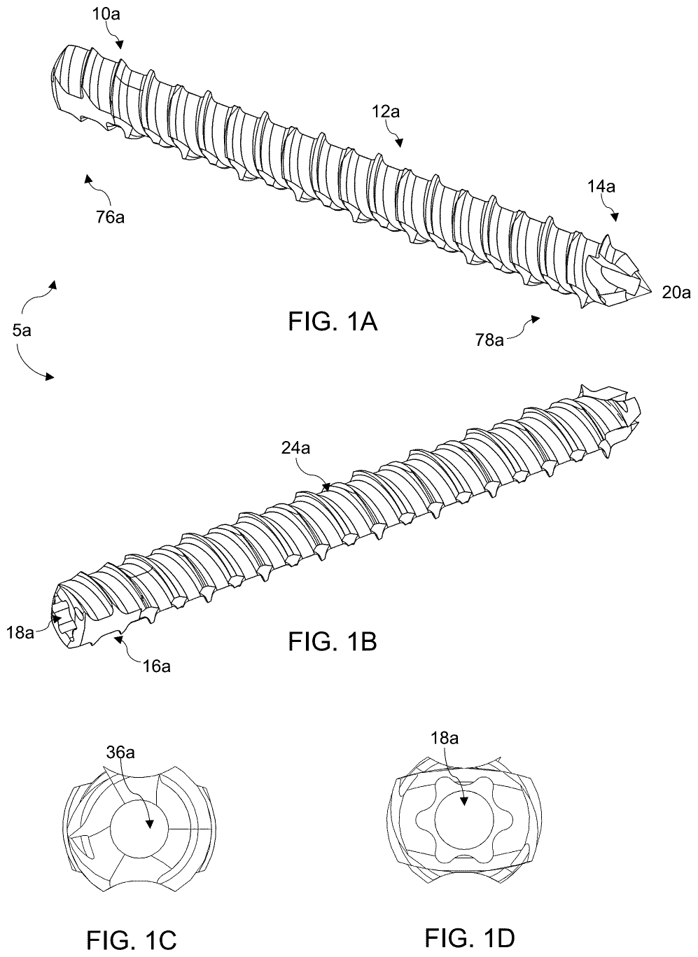

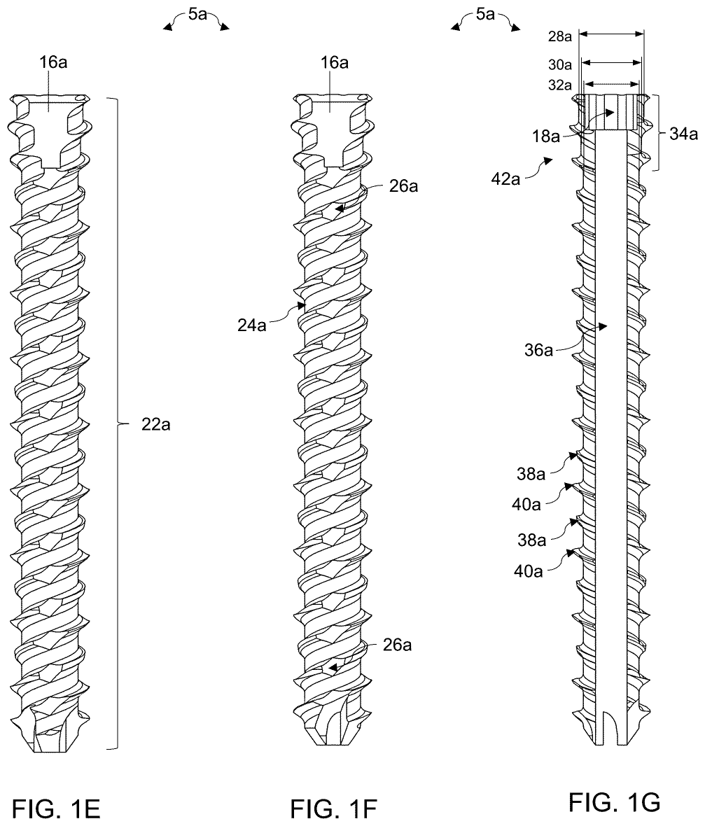

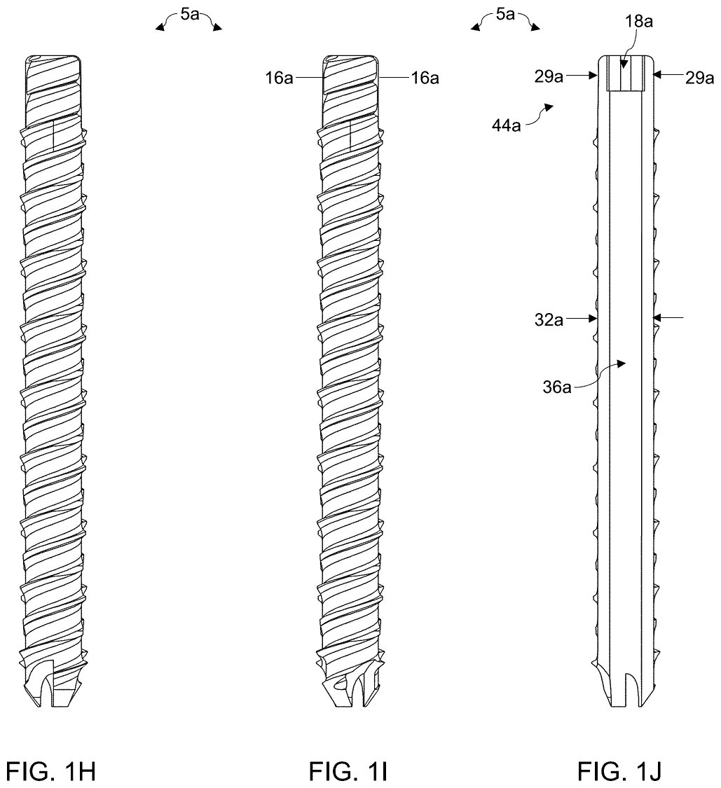

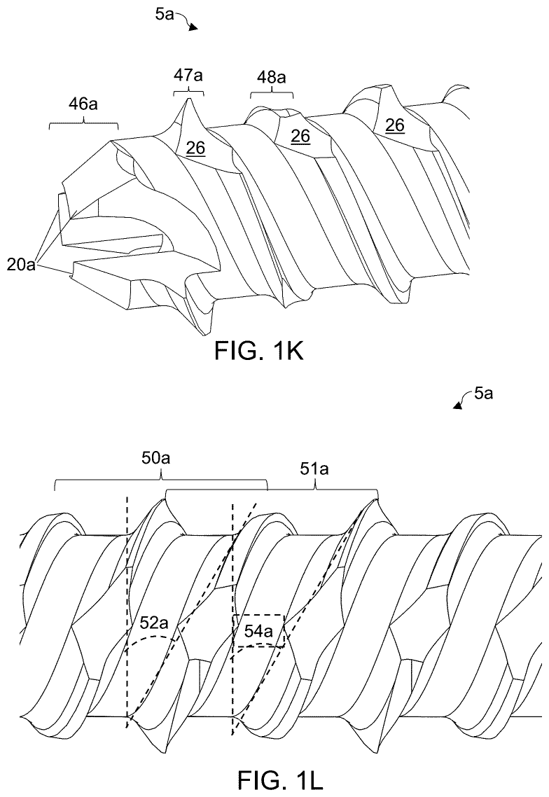

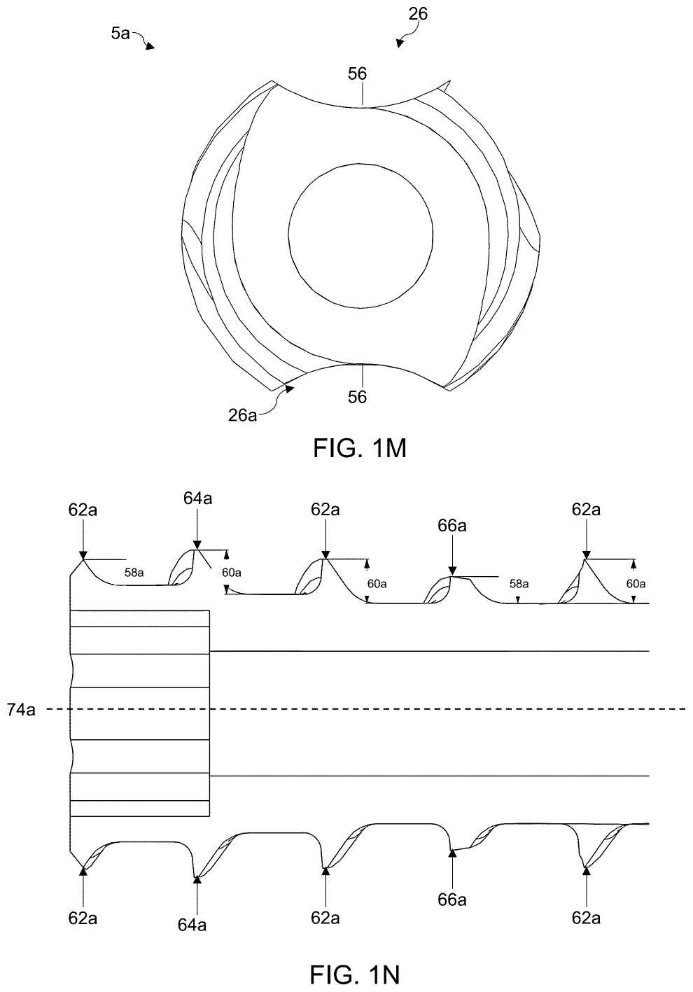

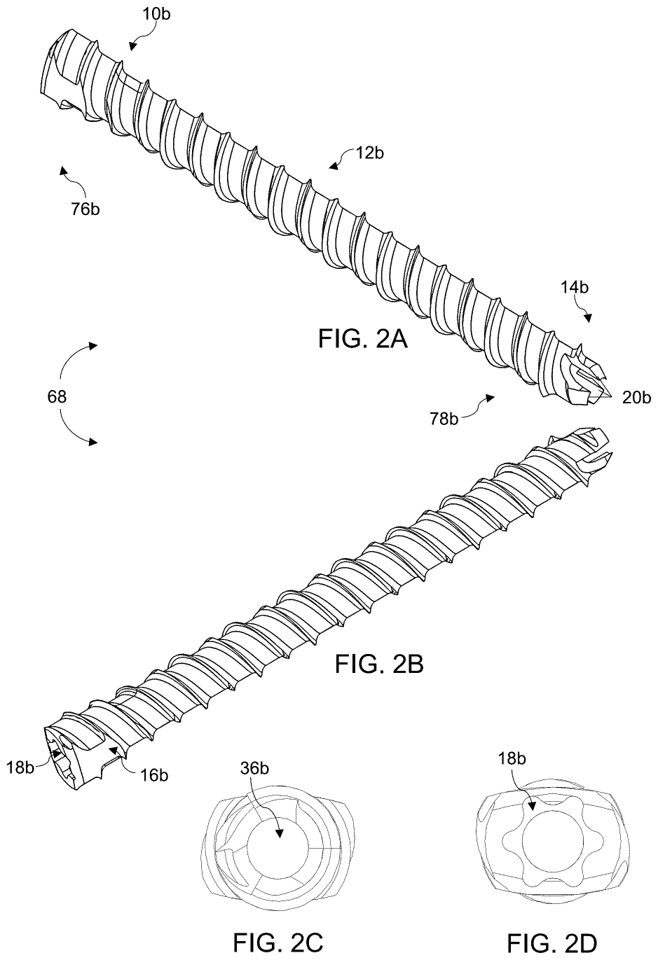

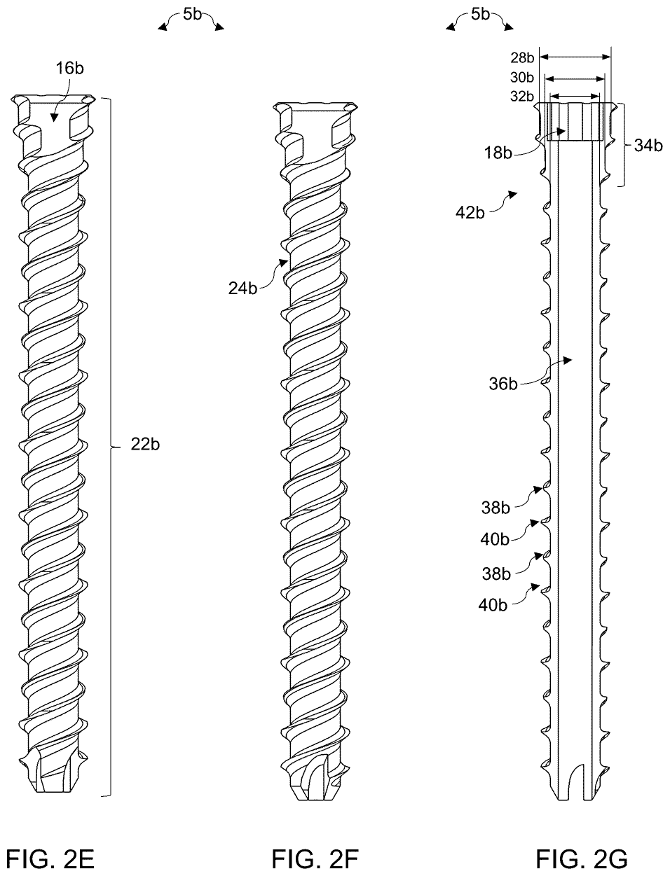

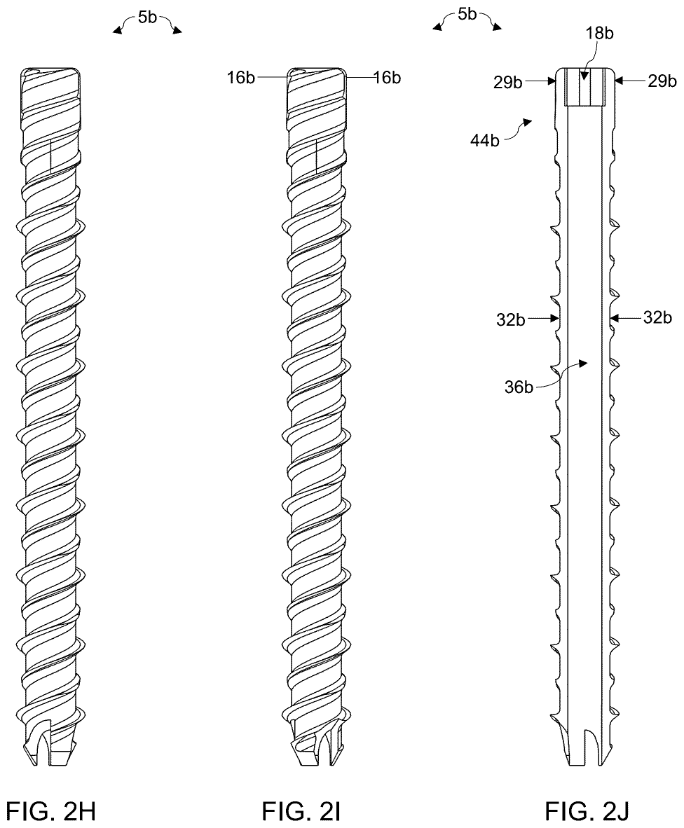

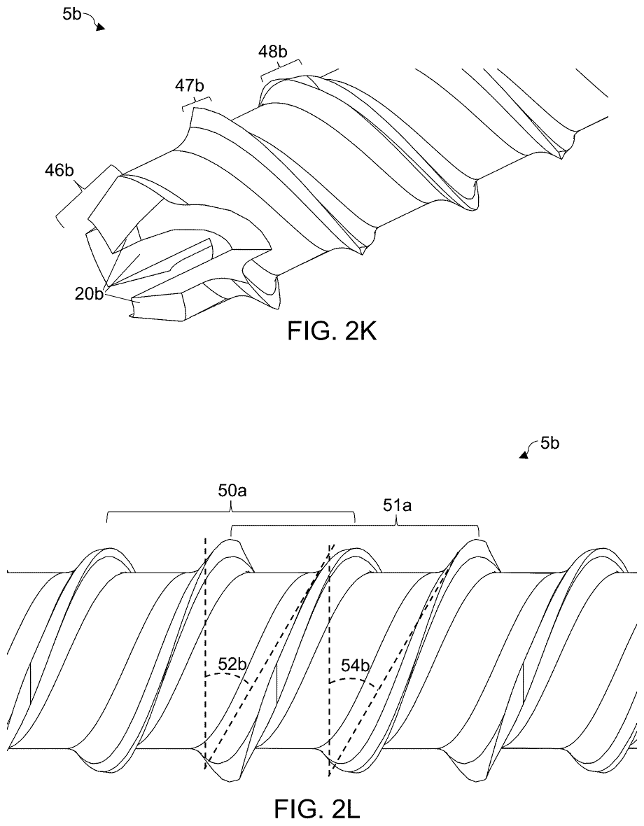

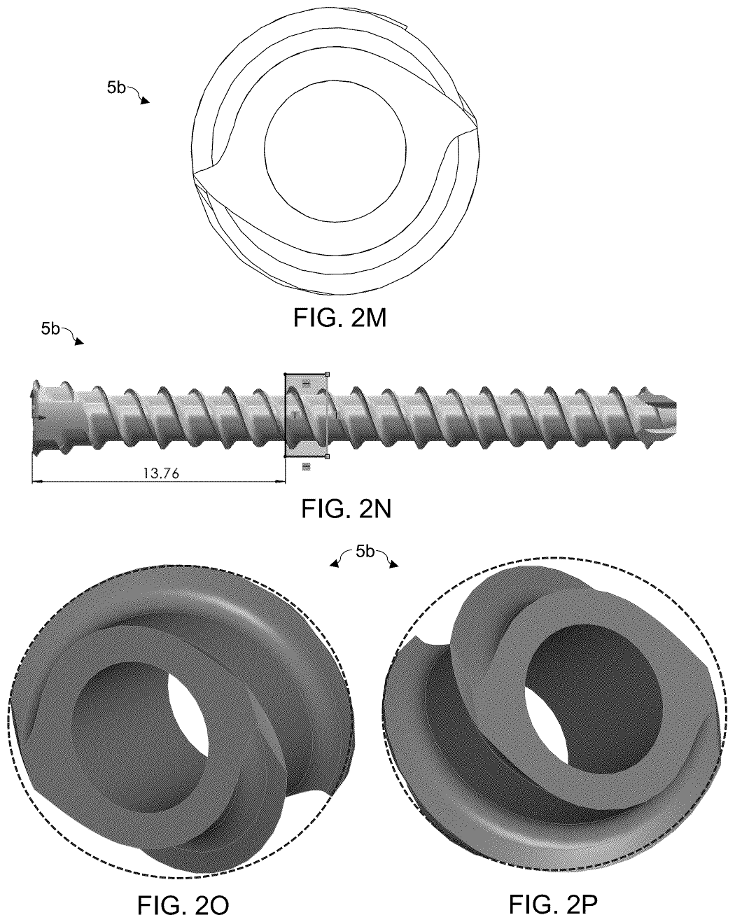

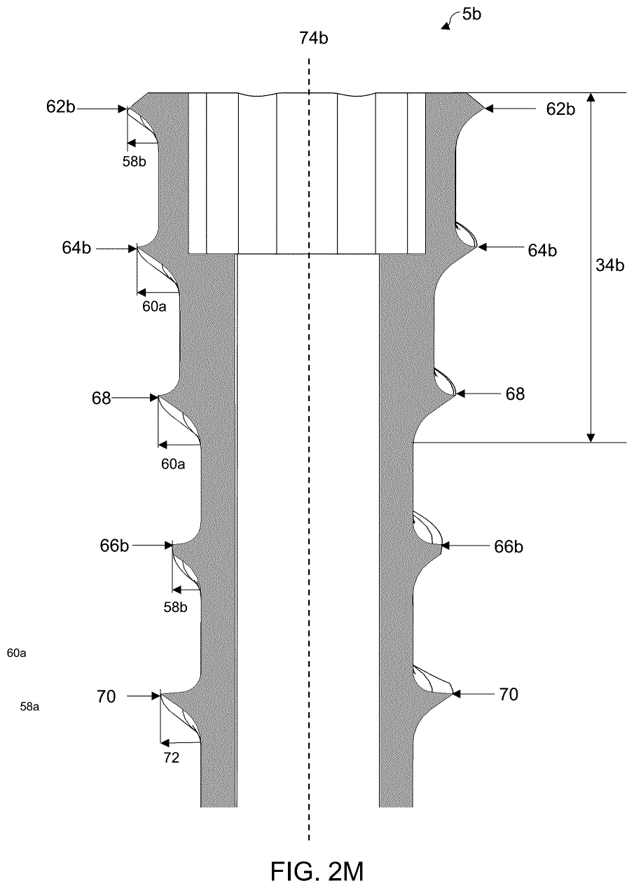

FIGS. 1A-1N and 2A-2M depict various plan views and magnified views of a first and second embodiment fixation screws 5a, 5b. The fixation screws 5a, 5b comprise a first end 76a, 76b, a second end 78a, 78b, and a longitudinal axis 74a, 74b. The fixation screws 5a, 5b further comprises a shaft 24a, 24b, a first helical thread 38a, 38b and a second helical thread 40a, 40b, and a total fixation screw length 22a, 22b. The shaft 24a, 24b comprises a first portion 10a, 10b, a second portion 12a, 12b, and a third portion 14a, 14b. The first portion 10a, 10b is disposed or positioned at or near the first end 76a, 76b. The third portion 14a, 14b is disposed or positioned at or near the second end 78a, 78b. The second portion 12a, 12b is disposed or positioned between the first portion 10a, 10b and the third portion 14a, 14b.

The total fixation screw length 22a, 22b may comprise 30 mm to 50 mm; the total fixation screw length may comprise 35 mm to 45 mm; the total fixation screw length 22a, 22b may comprise 35 mm to 40 mm; the total fixation screw length 22a, 22b may comprise at least 30 mm or greater; and the total fixation screw length 22a, 22b may comprise at least 35 mm or greater. In one exemplary embodiment, the total fixation screw length 22a, 22b is at least 35 mm. Alternatively, the total fixation screw length 22a, 22b may comprise 5 mm to 60 mm; the total fixation screw length 22a, 22b may comprise 8 mm to 60 mm; the total fixation screw length 22a, 22b may comprise 10 mm to 60 mm; the total fixation screw length 22a, 22b may comprise 12 mm to 60 mm; the total fixation screw length 22a, 22b may comprise 16 mm to 60 mm; the total fixation screw length 22a, 22b may comprise at least 12 mm or greater; and/or the total fixation screw length 22a, 22b may comprise at least 16 mm or greater. The fixation screws may be offered additionally in 2, 3, 4 or 5 mm increments.

The first portion 10a, 10b of the fixation screw 5a, 5b comprises dual cross-sectional profiles or shapes 42a, 42b, 44a, 44b to easily distinguish orientation during imaging. Accordingly, the first portion 10a, 10b of the fixation screw 5a, 5b comprises a first portion length 34a, 34b, a first cross-sectional profile or shape 42a, 42b and a second cross-sectional profile or shape 44a, 44b. The second cross-sectional profile 44a, 44b is perpendicular to the first cross-sectional profile 42a, 42b. Alternatively, the first portion 10a, 10b of the shaft 24a, 24b comprises a first cross-sectional shape 42a, 42b along the longitudinal axis 74a, 74b and a second cross-sectional shape 44a, 44b along the longitudinal axis 74a, 74b, the second cross-sectional shape 44a, 44b is perpendicular to the first cross-sectional shape 42a, 42b, the first cross-sectional shape 42a, 42b is different than the second cross-sectional shape 44a, 44b.

In one embodiment, the first cross-sectional shape or profile 42a, 42b comprises non-uniform shape and the second cross-sectional shape or profile 44a, 44b comprises a uniform shape. The non-uniform shape comprises a tapered shape and the uniform shape comprises a cylindrical or circular. The non-uniform shape is non-uniform along the first portion length 34a, 34b, and the uniform shape is uniform along the first portion length 34a, 34b.

In another embodiment, the first cross-sectional shape or profile 42a, 42b of the first portion 10a, 10b comprises a first cross-sectional diameter 28a, 28b, 30a, 30b and the first portion 10a, 10b comprises the second cross-sectional shape 44a, 44b and a second-cross-sectional diameter 29a, 29b, and the second portion 12a, 12b of the shaft 24a, 24b comprises a second portion shaft diameter 32a, 32b, the second cross-sectional diameter 29a, 29b substantially matches, matches and/or is the same as the second portion shaft diameter 32a, 32b. The first cross-sectional diameter 28a, 28b, 30a, 30b does not match, does not substantially match and/or is different than the second portion shaft diameter 32a, 32b.

The first portion length 34a, 34b may comprise 3.5 mm to 4.5 mm; the first portion length 34a, 34b may comprise 3.75 mm to 4.25 mm; the first portion length 34a, 34b may comprise 3.75 mm to 4.25 mm; the first portion length 34a, 34a may comprise at least 3.5 mm or greater; and/or the first portion length 34a, 34b may comprise at least 3.75 mm or greater. Accordingly, the first portion length 34a, 34b at least 10% to 20% of total fixation screw length 22a, 22b; the first portion length 34a, 34b at least 10% to 18% of total fixation screw length 22a, 22b the first portion length 34a, 34b at least 10% to 15% of total fixation screw length 22a, 22b; the first portion length 34a, 34b at least 10% of total fixation screw length 22a, 22b; and/or the first portion length 34a, 34b less than 20% of total fixation screw length 22a, 22b.

The first portion 10a, 10b may further comprise a driver socket 18a, 18b. The driver socket 18a, 18b may comprise a driver socket shape. The driver socket shape may comprise a slotted shape, a Phillips shape, a square shape, a hexagonal or hexalobular shape, a Torx shape, a hexagonal or hexalobular security shape, a Tri-wing shape, a Posidriv shape, a Clutch shape, a Spanner shape and/or any combination thereof. The driver socket shape may be sized and configured to receive a slotted driver, a Phillips driver, a square driver, a hexagonal or hexalobular driver, a Torx driver, a hexagonal or hexalobular security driver, a Tri-wing driver, a Posidriv driver, a Clutch driver, a Spanner driver and/or any combination thereof.

The first cross-sectional shape or profile 42a, 42b of the first portion 10a, 10b comprises a first cross-sectional diameter 28a, 28b, 30a, 30b that is non-uniform. The first cross-sectional diameter 28a, 28b, 30a, 30b may include at least 2.5 mm to 4 mm; first cross-sectional diameter 28a, 28b, 30a, 30b may include at least 2.5 mm to 3.5 mm; and/or the first cross-sectional diameter 28a, 28b, 30a, 30b may include at least 3.0 mm to 3.5 mm. In one embodiment, the first cross-sectional diameter 28a, 28b, 30a, 30b may change from 3.5 mm down to 3 mm along the first portion length 34a, 34b. Alternatively, the first cross-sectional diameter 28a, 28b, 30a, 30b may change from 3.5 mm down to 3 mm along the first portion length 34a, 34b in 0.25 mm increments. In another embodiment, the first cross-sectional diameter 28a, 28b, 30a, 30b may change from 3.5 mm down to 2.5 mm along the first portion length 34a, 34b. Alternatively, the first cross-sectional diameter 28a, 28b, 30a, 30b may change from 3.5 mm down to 2.5 mm along the first portion length 34a, 34b in 0.50 mm increments.

The first portion 10a, 10b comprises the second cross-sectional shape 44a, 44b and a second-cross-sectional diameter 29a, 29b that is uniform. The second-cross-sectional diameter 29a, 29b may match, substantially match the shaft diameter 32a, 32b in the second portion 12a, 12b. The shaft diameter 32a, 32b may comprise a range of 2 mm to 4 mm; the shaft diameter 32a, 32b may comprise a range of 2.5 mm to 3.5 mm; the shaft diameter 32a, 32b may comprise a range of 2.5 mm to 3 mm; and/or the shaft diameter 32a, 32b may comprise a range of at least 2.5 mm or greater. In one embodiment, the first fixation screw 5a may comprise a first shaft diameter 32a and the second fixation screw 5b may comprise a second shaft diameter 32b. The first shaft diameter 32a may comprise at least 3 mm or greater and the second shaft diameter 32b may comprise at least 2.5 mm or greater.

The first portion 10a, 10b, the second portion 12a, 12b, and the third portion 14a, 14b of the shaft 24a, 24b an outer diameter and/or outer surface shape. The outer diameter or outer surface shape may comprise an oval, an ellipse and/or a circle. The outer diameter or outer surface shape may comprise a non-circular shape as shown in FIGS. 1M, 2M-2N, 3E and 5E.

The fixation screws 5a, 5b comprise the first helical thread 38a, 38b and a second helical thread 40a, 40b. The first helical thread 38a, 38b including a first helical thread width 48a, 48b, a first thread pitch 50a, 50b, a first helix angle 52a, 52b, a first thread depth 58a, 58b, and a first thread diameter 66a, 66b. The second helical thread 40a, 40b including a second helical thread width 47a, 47b, a second thread pitch 51a, 51b, a second helix angle 54a, 54b, a second thread depth 60a, 60b, 72, and a second thread diameter 62a, 62b, 64a, 64b, 68, 70. The first helical thread 38a, 38b and the second helical thread 40a, 40b are disposed between the first end 76a, 76b and the second end 78a, 78b of and along the longitudinal axis 74a, 74b. In one embodiment, the first helical thread 38a, 38b and the second helical thread 40a, 40b are disposed between the first end 76a, 76b and alternate uniformly along the longitudinal axis 74a, 74b. In another embodiment, first helical thread 38a, 38b and the second helical thread 40a, 40b are disposed between the first end 76a, 76b and the second end 78a, 78b, and alternate uniformly along second portion 12a, 12b of the shaft 24a, 24b. In another embodiment, first helical thread 38a, 38b and the second helical thread 40a, 40b are disposed between the first end 76a, 76b and second end 78a, 78b and alternate uniformly along the second portion 12a, 12b and the third portion 14a, 14b of the shaft 24a, 24b. In another embodiment, the first helical thread 38a, 38b and the second helical thread 40a, 40b are disposed between the first end 76a, 76b and alternates non-uniformity in the first portion 10a, 10b of the shaft 24a, 24b.

In another embodiment, the first helical thread width 47a, 47b and the second helical thread width 48a, 48b comprise different widths. The first helical thread width 47a, 47b and the second helical thread width 48a, 48b comprise different widths. The first helical thread width 47a, 47b is smaller than the second helical thread width 48a, 48b. In another embodiment, the first helical thread depth 58a, 58b and the second helical thread depth 60a, 60b, 72 comprise different thread depths. The first helical thread depth 58a, 58b and the second helical thread depth 60a, 60b, 72 comprise the same thread depths. The first helical thread depth 58a, 58b is smaller than the second helical thread depth 60a, 60b, 72.

In another embodiment, the first helical thread depth 58a, 58b and the second helical thread depth 60a, 60b, 72 alternates uniformly between the first end 76a, 76b and the second end 78a, 78b. In another embodiment, the first helical thread depth 58a, 58b and the second helical thread depth 60a, 60b, 72 alternates non-uniformly between the first end 76a, 76b and the second end 78a, 78b. The first helical thread depth 58a, 58b and the second helical thread depth 60a, 60b, 72 alternates uniformly within the second portion 12a, 12b of the shaft 24a, 24b. The first helical thread depth 58a, 58b and the second helical thread depth 60a, 60b, 72 alternates uniformly within the second portion 12a, 12b and the third portion 14a, 14b of the shaft 24a, 24b.

In another embodiment, the first helical thread 38a, 38b and/or the second helical thread 40a, 40b repeat in the first portion 10a, 10b of the shaft 24a, 24b. Alternatively, the second helical thread 40a, 40b repeats in the first portion 10a, 10b of the shaft 24a, 24b. In another embodiment, the second helical thread 40a, 40b repeats in the first portion 10a, 10b of the shaft, and the second helical thread 40a, 40b alternates patterns with the first helical thread 38a, 38b within the second portion 12a, 12b and the third portion 14a, 14b of the shaft 24a, 24b.

In another embodiment, the first helical thread diameter 66a, 66b and/or the second helical thread diameter 62a, 62b, 64a, 64b, 68, 70 may change in the first portion 10a, 10b of the shaft 24a, 24b. The first helical thread diameter 66a, 66b and/or the second helical thread diameter 62a, 62b, 64a, 64b, 68, 70 may decrease from the top thread diameter 62a (the highest diameter), 62b to the bottom thread diameter 64a, 64b, 68 (the lower or smaller diameter) in the first portion 10a, 10b of the shaft 24a, 24b. The first portion 10a, 10b may comprise at least three helical threads that include only the first helical thread 38a, 38b, the second helical thread 40a, 40b, and/or the first helical thread 38a, 38b and the second helical thread 40a, 40b. The first helical thread diameter 66a, 66b and/or the second helical thread diameter 62a, 62b, 64a, 64b, 68, 70 may alternate uniformly within the second portion 12a, 12b and/or the third portion 14a, 14b of the shaft 24a, 24b.

The first helical thread diameter 66a, 66b may comprise a range 3.5 mm to 4.75 mm; the first helical thread diameter 66a, 66b may comprise a range 3.5 mm to 4.5 mm; the first helical thread diameter 66a, 66b may comprise a range 3.75 mm to 4.25 mm; the first helical thread diameter 66a, 66b may comprise at least 3.75 mm or greater; and/or the first helical thread diameter 66a, 66b may comprise a range at least 4 mm or greater. The second helical thread diameter 62a, 62b, 64a, 64b, 68, 70 may comprise a range of 3 mm to 4.5 mm; the second helical thread diameter 62a, 62b, 64a, 64b, 68, 70 may comprise a range of 3 mm to 4.25 mm; the second helical thread diameter 62a, 62b, 64a, 64b, 68, 70 may comprise a range of 3.20 mm to 4.25 mm; the second helical thread diameter 62a, 62b, 64a, 64b, 68, 70 may comprise at least 3 mm or greater; the second helical thread diameter 62a, 62b, 64a, 64b, 68, 70 may comprise a range of 3.5 mm or greater.

In another embodiment, the first helical thread 38a, 38b and/or the second helical thread 40a, 40b may comprise a thread shape as shown in 1M, 2M, and 2M-2P. The thread shape may comprise a oval, an ellipse, a circle, and/or any combination thereof. The thread shape may comprise a semi-oval, a semi-ellipse, a semi-circle, and/or any combination thereof. The thread shape may comprise a portion of an oval, a portion of an ellipse, apportion of a circle, and/or any combination thereof.

The first and second fixation screws 5a, 5b may comprise one or more facets 16a, 16b. The one or more facets 16a, 16b may be disposed onto a first portion 10a, 10b of the shaft 24a, 24b. The one or more facets 16a, 16b may be disposed onto the first cross-sectional shape or profile 42a, 42b of the first portion 10a, 10b. The one or more facets 16a, 16b may be disposed onto a portion of the first portion 10a, 10b of the shaft 24a, 24b and/or disposed along the longitudinal axis 74a,74b or the first portion length 34a, 34b. The one or more facets 16a, 16b may include a flat, planar surface.

The first and second fixation screws 5a, 5b may comprise a plurality of scallops 26. The plurality of scallops 26 being sized and configured to receive a second bone fixation screw or the thread diameter of the second bone fixation screw. In one embodiment, the plurality of scallops 26 may be disposed along the longitudinal axis 74a, 74b of the first and/or second fixation screws 5a, 5b between the first end 76a, 76b and the second end 78a, 78b. In another embodiment, the plurality of scallops 26 may be disposed on the first helical thread 38a, 38b and/or the second helical thread 40a, 40b along the longitudinal axis 74a, 74b of the first and/or second fixation screws 5a, 5b between the first end 76a, 76b and the second end 78a, 78b. In another embodiment, the plurality of scallops 26 may be disposed on the first helical thread 38a, 38b and/or the second helical thread 40a, 40b along the longitudinal axis 74a, 74b of the first and/or second fixation screws 5a, 5b within the second portion 12a, 12b and/or the third portion 14a, 14b of the shaft 24a, 24b. The plurality of scallops 26 comprises a scallop surface 56, the scallop surface 56 may comprise a concave shape. The scallop surface 56 may be sized and configured to receive a portion of a second bone fixation screw and/or a second helical thread diameter. The plurality of scallops 26 are positioned on the same side as the one or more facets 16a, 16b.

The first and second fixation screws 5a, 5b may comprise a lumen 36a, 36b. The lumen 36a, 36b is disposed in the center of the fixation screws 5a, 5b, and extends from a first end 76a, 76b through the second end 78a, 78b. The lumen 36a, 36b extends along the longitudinal axis 74a, 74b between the first end 76a, 76b and the second end 78a, 78b. The lumen 36a, 36b is sized and configured to receive a portion of K-wire or other guide wires. The lumen 36a, 36b is sized and configured to receive a portion of 1.6 mm K-wire or other guide wires.

### Manufacturing of the Fixation Screws

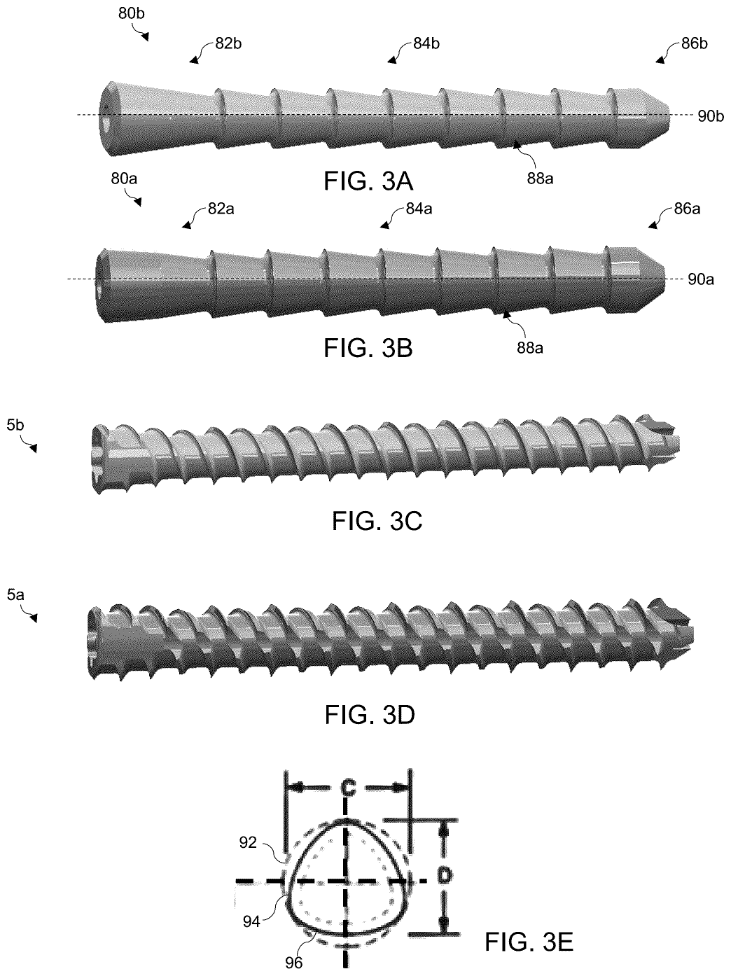

FIGS. 3A-3D depict side views of each fixation screw blank 80a, 80b and the fixation screw 5a, 5b final product. More specifically, FIG. 3A represents a 3.5 mm screw blank 80b, FIG. 3B represents a 4.0 mm screw blank 80a, FIG. 3C represents a 3.5 mm fixation screw with double helix threads 5b, and FIG. 3D represents a 4.0 mm fixation screw with double helix threads 5a. The manufacturing of the fixation's screws 5a, 5b may comprise a first shaft blank 80a and second shaft blank 80b. The shaft blanks 80a, 80b comprises a first portion 82a, 82b, a second portion 84a, 84b and a third portion 86a, 86b. The second portion 84a, 84b is positioned between the first portion 82a, 82b and the second portion 86a, 86b. The first portion 82a, 82b is near or adjacent to the first end 76a, 76b, and the third portion 86a, 86b is positioned near or adjacent to the second end 78a, 78b. The first portion 82a, 82b comprise a tapered shape. The third portion 86a, 86b comprise a tapered shape. The second portion 84a, 84b comprises a plurality of tapered cones or truncated cones 88a, 88b that contact each other and are stacked. In another embodiment, first shaft blank 80a and second shaft blank 80b of the first and second fixation screws 5a, 5b comprise a first and second cross-sectional blank shape (not shown), the first and second cross-sectional blank shape is along the longitudinal axis 90a, 90b. The first and second cross-sectional blank shape is non-uniform along the longitudinal axis 90a, 90b.

The first shaft blank 80a and second shaft blank 80b will be manufactured into thread-forming or self-tapping fixation screws 5a, 5b. Such thread-forming or self-tapping feature will allow the fixation screws 5a, 5b to form its own threads into the mating bone material. This is advantageous because it can eliminate the need for pre-drilling holes or tapping, saving assembly, production costs, and instrumentation costs. Also, such thread forming creates a stable and secure connection due to its resistance to vibration loosening because it forms its own threads while installing resulting in no gaps between male and female threads. The double helix thread form progresses over the entire screw length and over the slightly flared head to prove the user with feedback that is similar to a “tightening feel” even though the screw is a non-tightening fixation screw design in that it does not compress the bone halves together by itself.

In another embodiment, first shaft blank 80a and second shaft blank 80b may be manufactured via the trilobular thread forming technique as shown in FIG. 3E. The trilobular thread forming technique allows each thread lobe 92, 94, 96 of the screw to move through the pilot hole in the nut material, forming and work hardening the nut thread material, producing an uninterrupted grain flow. The tri-lobe technique further enables the thread lobes 92, 94, 96 to form an internal thread. The fixation screws 5a, 5b may further comprise a tapered tip or a third portion 14a, 14b with a tapered shape that is commonly referred to as the lead threads as shown in FIGS. 3C-3D. The tapered shape allows for lower threading or installation torque during initial thread forming into the targeted substrate. Also, the trilobular thread forming technique can produce a non-circular shape in the shaft of the fixation screws 5a, 5b. The tri-lobe shape is key to allowing the remaining threads to form threads with the lowest amount of installation or threading torque.

### Stabilizing the Capital Fragment Method

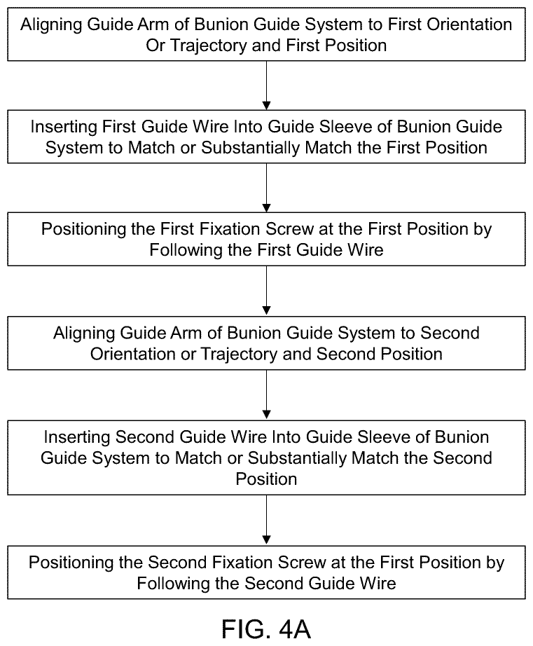

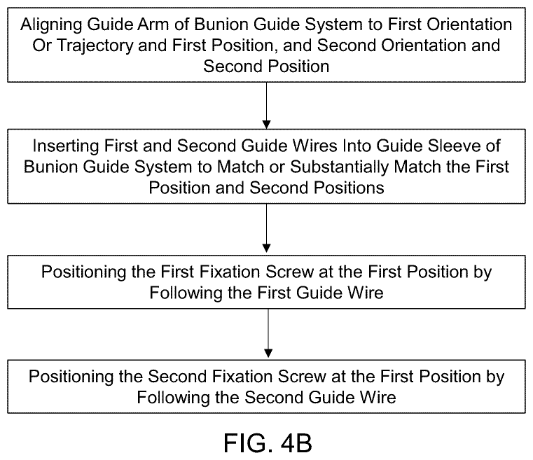

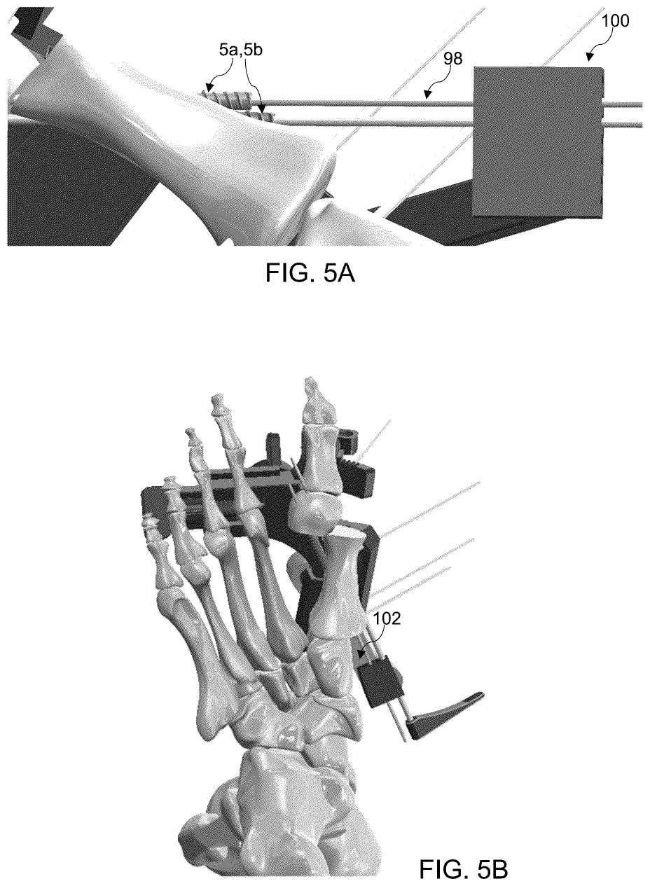

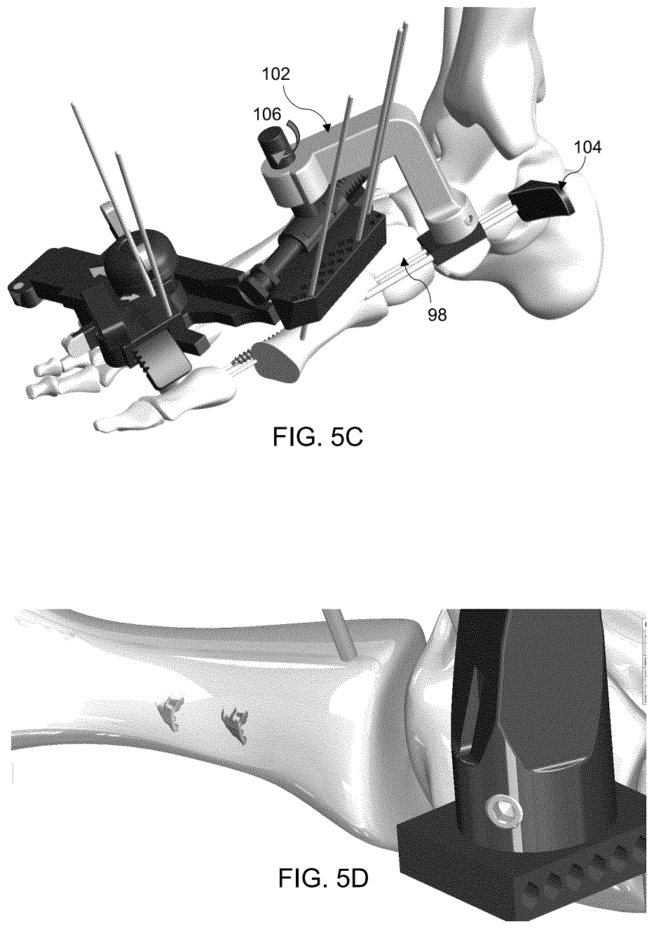

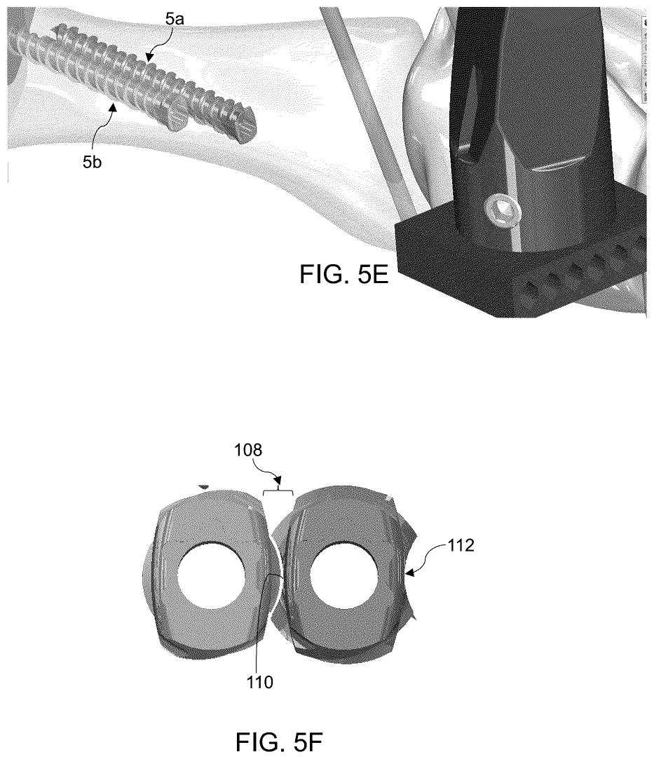

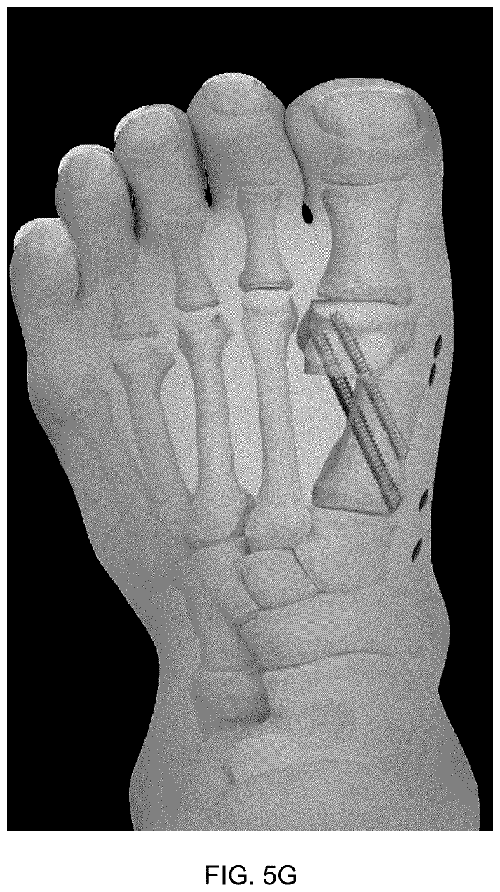

FIGS. 4A-4B and 5A-5F depict one or more embodiments of the method of stabilizing the capital fragment using a bunion guide system. In one embodiment, the method of stabilizing the capital fragment comprises: aligning the guide arm of the bunion guide system to a first orientation or trajectory and a first position; inserting a first guide wire into a guide sleeve of bunion guide system to match, follow or align or substantially match, follow or align to the first orientation or trajectory; positioning a first fixation screw at the first position while translating or following along the first guide wire; aligning the guide arm of the bunion guide system to a second orientation or trajectory and a second position; inserting a second guide wire into a guide sleeve of bunion guide system to match, follow or align or substantially match, follow or align to the second orientation or trajectory; positioning a second fixation screw at the second position while translating or following along the second guide wire. The method of stabilizing may further comprise creating a first bone hole and/or creating a second bone hole. The method of stabilizing may further comprise securing the first fixation screw and/or securing the second fixation screw.

In another embodiment, the method of stabilizing the capital fragment comprises: aligning a guide arm of the bunion guide system to a first orientation or trajectory and a first position and to a second orientation or trajectory and a second position; inserting a first guide wire into a guide sleeve to follow or align with the first trajectory and inserting a second guide wire into a guide sleeve to follow or align the with the second trajectory; positioning the first fixation screw at the first position and the second fixation screw at the second position. The method of stabilizing may further comprise creating a first bone hole and a second bone hole. The method of stabilizing may further comprise securing the first fixation screw to the first position and the second fixation screw to the second position.

In one embodiment, the method of stabilizing the capital fragment comprises the step of aligning the guide arm to first and/or second trajectory for a first and/or second position. The bunion guide system will hold the final positioning (fixed in space relative to desired final position) of the capital fragment, which the final positioning is in a more aligned anatomical trajectory for the one or more fixation screws to be secured. The fixation guide arm assembly will be used to stabilize/secure the capital fragment. Once three or more planes of correction have been achieved to obtain the final positioning of the capital fragment, the guide arm of the bunion guide arm assembly can be rotated 0 degrees to 60 degrees to achieve the proper trajectory of the one or more fixation screws to reach the first and/or second positions. The guide arm may be locked to the first and/or second trajectory(ies). The first position may comprise proximal position on the capital fragment and the second position may comprise distal position on the capital fragment.

In another embodiment, the method of stabilizing the capital fragment comprises the step of inserting a first and/or second guide wire into a guide sleeve of the bunion guide system to reach the first and/or second positions on the capital fragment. A first guidewire or K-wire may be inserted through the drill sleeve and/or sleeve jig of the fixation guide arm assembly to be introduced or advanced along the proximal aspect of the body of the first metatarsal to follow or align or substantially follow or align with the intended first trajectory to arrive at the first position. The guidewire trajectory is confirmed with imaging to ensure that it matches or substantially matches the intended trajectory position. The physician should ensure that there is adequate bone purchase with the first guide wire to provide stabilization. The first placement or positioning of the first guide wire or K-wire for the first screw may traverse both cortices of the proximal metatarsal prior to penetrating the capital fragment. K-wire or guide wire may comprise standard point tip or a threaded tip. Confirm proper placement of the guide wires or K-wires via imaging assistance.

Repeat the above steps for the second guide wire to reach the second position. The second guidewire may be inserted through the drill sleeve and/or sleeve jig to enter distal to the proximal or first guidewire in a parallel fashion. The second guidewire may be positioned at or near the central aspect of the capital fragment to ensure fixation. The K-wire or guidewire may include one or more different tips such as threaded or non-threaded, diamond, trocar, slotted trocar, notched trocar, Mendin, and/or hollow notched trocar. The first and second position may comprise medial proximal first metatarsal cortex and into capital fragment and/or proximal lateral first metatarsal cortex. Confirm proper placement of the guidewires or K-wires via imaging assistance.

In another embodiment, the method of stabilizing the capital fragment comprises the step of determining fixation screw length and/or diameter. The step of determining screw fixation screw length and/or fixation screw diameter comprises measuring the depth of the guide wire insertion through the first metatarsal through the capital fragment. Tools or measuring devices known in the art may be used to obtain a desired length and/or diameter. Also, the guidewires may comprise measurement notches to obtain the appropriate depth. The depth measurements help select the proper fixation screw length and/or diameter for the patient's anatomy. Screw lengths should be carefully measured to obtain as much length and bony fixation as possible.

In another embodiment, the method of stabilizing the capital fragment comprises the step of creating a first and/or second bone hole. A cannulated drill bit may be advanced over the first and/or second guidewires. Once drilling commences, the drilled hole length can be monitored to reach the desired depth via imaging. The cannulated drill bit may further comprise depth markings to assist the physician.

In another embodiment, the method of stabilizing the capital fragment comprises the step of positioning the first and/or second fixation screws to the first and/or second positions on the capital fragment. The selected fully threaded screws are advanced over the one or more guidewires (e.g., the first and second guidewires) and across the osteotomy site to the first and/or second positions. If possible, the proximal fixation screw is placed first prior to the second screw into the capital fragment. If the second fixation screw requires placement or positioning distance of 3 mm or less from the first fixation screw, the first fixation screw should be rotated to allow the one or more scallops to be faced towards the second fixation screw as shown in FIG. 5E. The one or more fixation screws may comprise 3.5 mm or 4 mm fully threaded, headless non-compression screws with beveled/tapered tips to secure the osteotomy site. The bicortical fixation without soft tissue disruption of the vascularized tissues secures the osteotomy and permits bony remodeling laterally. Verify positioning and/or placement of the first and/or second fixation screws via imaging. The first end of the first and/or second fixation screws should be at or below the patient's skin surface.

FIG. 5A-5B illustrates a plantar view of the foot to show the “close proximity” of the fixation screws 5a, 5b if required by the surgeon due to a small or tight anatomy. Surgical fixation of the bunion deformity is achieved via the stabilization of the capital fragment with internal medially placed fixation screws 5a, 5b. The guidance of the k-wires or guidewires 98 for the fixation screws is achieved via the guide arm 102, drill sleeve 104 and guide arm base 100. The guide arm 102 can rotate 106 from 0 degrees up to 75 degrees as shown in FIG. 5C. FIG. 5D shows one embodiment of an angled insertion into the metatarsal body and the low-profile protrusion of the double-scalloped flared head of the fixation screws 5a, 5b.

FIGS. 5E-5G further illustrates the close proximity of a first screw 5a and a second screw 5b. The first fixation screw 5a is proximally placed and the second fixation screw 5b is distally placed relative to the first fixation screw 5a. The plurality of scallops 26 of the first fixation screw 5a should face towards the second fixation screw 5b, the second fixation screw 5b can be positioned against the plurality of scallops 26 (e.g. mated) and/or spaced apart 108 at least 5 mm or less. Alternatively, it may be spaced apart 108 at least 3 mm or less, and/or 0 mm to 5 mm. As previously described herein, the fixation screws 5a, 5b may comprise a first plurality of scallops 110 and a second plurality of scallops 112. The first plurality of scallops 110 may be diametrically opposed to the second plurality of scallops 112. Alternatively, the first plurality of scallops 110 may be positioned 180 degrees apart from the second plurality of scallops 112. The reduced spacing 108 of the fixation screws is an improvement over traditional fixation screws, where the traditional fixation screws are placed 6 mm or greater for bone stability and to ensure that the thread profiles of the traditional fixation screws do not contact.

### Fixation Screw Sterile Kit

In one embodiment, the bunion guide system may comprise a fixation screw sterile packaged kit. The sterile packaged kit includes a packaging tray, one or more fixation screws, one or more guidewires or k-wires; and/or one or more cannulated drill bits. The sterile packaged kit may further comprise depth gages or tools. The fixation screw sterile packaged kit maintains the fixation screws and its respective instruments sterile after sterilization of the sterile packaged kit.

In one embodiment, a method of packaging a fixation screw system comprises the steps of: inserting at least one fixation screw into a first compartment of the packaging tray; inserting or securing the one or more guidewires or k-wires into a second compartment of the packaging tray; inserting the cannulated drill bit into a third compartment of the packaging tray. The packaging tray is enclosed by inserting an outer cover or lid over the packaging tray and sealing over the opening and flange of the packaging tray. The enclosed packaging tray and the fixation guide system therein are sterilized.

In another embodiment, a method of packaging a fixation screw system comprises the steps of: inserting at least one fixation screw onto a first region of the packaging insert or die cut card; inserting or securing the one or more guidewires or k-wires into a second region of the packaging insert card; inserting the cannulated drill bit into a third region of the packaging insert card; positioning the assembled insert card into a packaging pouch; sealing the packaging pouch; sterilizing the packaging pouch. The insert or die cut card and/or packaging pouches may comprise materials known in the art. The one or more regions of the packaging insert or die cut cards may comprise at least one securing or retention mechanism. The at least one securing or retention mechanism may comprise tabs, slots, protective folds, straps and/or any combination thereof.

### Additional Embodiments

A bone fixation screw system comprising: a first bone fixation screw, the first bone fixation screw comprises a first shaft, the first shaft comprising a first shaft longitudinal axis, first end, a second end, and first total shaft length; a first thread series, the first thread is disposed between the first end and the second end of the shaft and along the shaft longitudinal axis; and a second bone fixation screw, the second bone fixation screw comprises a second shaft, the second shaft comprising a second shaft longitudinal axis, first end, a second end, and second total shaft length; a second thread series, the second thread is disposed between the first end and the second end of the second shaft and along the second shaft longitudinal axis; and a plurality of scallops, the plurality of scallops including a scallop shape and a scallop depth, the plurality of scallops being sized and configured to receive a portion of the first bone fixation screw.

The first and second thread series comprises a helical thread. At least a portion of the first or second thread series comprises cutting threads. The first thread series comprises a first thread depth, and the second thread series comprises a second thread depth, the first thread depth is different than the second thread depth. The first thread series or set comprises a first helical thread and a second helical thread, and the second thread series comprises a third helical thread and a fourth helical thread.

Each of the first shaft and second shaft comprises a first portion, a second portion, and a third portion, the first portion adjacent to the first end, the second portion between the first portion and the third portion, the third portion adjacent to the second end, the first portion of the shaft comprises a first cross-sectional shape along the shaft longitudinal axis and a second cross-sectional shape along the shaft longitudinal axis, the second cross-sectional shape is perpendicular to the first cross-sectional shape, the first cross-sectional shape is different than the second cross-sectional shape. The first cross-sectional shape is circular and uniform along a first portion length, and the second cross-sectional is circular and non-uniform along the first portion length. The non-uniform along the first portion length comprises a taper. The first cross-sectional shape of the first portion comprises a first cross-sectional diameter and the second cross-sectional shape comprises a second-cross-sectional diameter, and the second portion of the shaft comprises a second portion shaft diameter, the first cross-sectional diameter substantially matches or matches the second portion shaft diameter. The first cross-sectional shape of the first portion comprises a first cross-sectional diameter and the second cross-sectional shape comprises a second-cross-sectional diameter, and the second portion of the shaft comprises a second portion shaft diameter, at least a portion of the second cross-sectional diameter is different the second portion shaft diameter. The first cross-sectional shape of the first portion comprises a first cross-sectional diameter and the second cross-sectional shape comprises a second-cross-sectional diameter, and the second portion of the shaft comprises a second portion shaft diameter, at least a portion of the second cross-sectional diameter is different than the second portion shaft diameter and the first cross-sectional diameter is the same or substantially the same as the second portion shaft diameter.

The plurality of scallops of the second bone fixation screw is disposed onto a portion of the first helical thread or a portion of the second helical thread. The plurality of scallops of the second bone fixation screw is disposed onto a portion of the first helical thread and a portion of the second helical thread. The plurality of scallops of the second bone fixation screw comprising a scallop depth, the scallop depth substantially matching or matching the first and/or second helical thread depth. The plurality of scallops comprising a scallop shape, the scallop shape including a concave shape.

The third portion of the first bone fixation screw and the third portion of the second bone fixation screw comprises a taper. The third portion of the first bone fixation screw and the third portion of the second bone fixation screw further comprises at least two cutting flutes. The first portion of the shaft for each of the first and/or second bone fixation screw comprises a first portion length, the first portion length comprising at least 10% of the total shaft length. The first portion of the shaft of each of the first and/or second bone fixation screw comprises a first portion length, the first portion length comprising a range of 10% to 20% of total shaft length.

A fixation screw system sterile kit comprising: a packaging tray, the packaging tray comprising body and a flange, the flange extends outwardly from a first end of the body, the body including plurality of compartments; a fixation screw system comprising a plurality of fixation screws and fixation screw instruments, the plurality of fixation screws and the fixation screw instruments are inserted into each of the plurality of compartments; a packaging lid or cover that is disposed over the packaging tray to create a sealed package that remains sterile within packaging tray after sterilization of the sealed package.

The plurality of fixation screws comprises a first fixation screw and a second fixation screw. The fixation screw instruments comprise one or more guidewire or k-wires, and one or more cannulated drill bits. The fixation screw instruments further comprise a depth gauge or depth tool.

A method of packaging a bunion guide system assembly comprises the steps of: inserting a first fixation screw into a first compartment of the packaging tray; inserting or securing second fixation screw into a second compartment of the packaging tray; securing the one or more guidewires or k-wires into a third compartment of the packaging tray; inserting a cannulated drill bit into a fourth compartment of the packaging tray; enclosing the packaging tray by inserting an outer cover or lid over the packaging tray and sealing over the opening and flange of the packaging tray to create a sealed package; and sterilizing the sealed package.

A bone fixation screw comprising: a shaft, the shaft comprising a shaft longitudinal axis, first end, a second end, total shaft length, a first portion, a second portion, and a third portion, the first portion adjacent to the first end, the second portion between the first portion and the third portion, the third portion adjacent to the second end; a first helical thread, the first helical thread including a first thread depth and a first thread pitch; and a second helical thread, the second helical thread including a second thread depth and a second thread pitch, the first helical thread and the second helical thread are disposed between the first end and the second end of the shaft and along the shaft longitudinal axis; and a plurality of scallops, the plurality of scallops being sized and configured to receive a second bone fixation screw.

The first helical thread and the second helical thread alternate along the shaft longitudinal axis. The first thread depth and the second thread depth are different. The first thread depth is larger than the second thread depth. The first helical thread or the second helical thread comprises a cutting thread.

The first portion of the shaft comprises a first cross-sectional shape along the shaft longitudinal axis and a second cross-sectional shape along the shaft longitudinal axis, the second cross-sectional shape is perpendicular to the first cross-sectional shape, the first cross-sectional shape is different than the second cross-sectional shape. The first cross-sectional shape is circular and uniform along a first portion length, and the second cross-sectional is circular and non-uniform along the first portion length. The first cross-sectional shape of the first portion comprises a first cross-sectional diameter and the second cross-sectional shape comprises a second-cross-sectional diameter, and the second portion of the shaft comprises a second portion shaft diameter, the first cross-sectional diameter substantially matches or matches the second portion shaft diameter. The non-uniform along the first portion length comprises a taper. The first cross-sectional shape of the first portion comprises a first cross-sectional diameter and the second cross-sectional shape comprises a second-cross-sectional diameter, and the second portion of the shaft comprises a second portion shaft diameter, at least a portion of the second cross-sectional diameter is different the second portion shaft diameter. The first cross-sectional shape of the first portion comprises a first cross-sectional diameter and the second cross-sectional shape comprises a second-cross-sectional diameter, and the second portion of the shaft comprises a second portion shaft diameter, at least a portion of the second cross-sectional diameter is different than the second portion shaft diameter and the first cross-sectional diameter is the same or substantially the same as the second portion shaft diameter.

The plurality of scallops is disposed onto a portion of the first helical thread or a portion of the second helical thread. The plurality of scallops is disposed onto a portion of the first helical thread and a portion of the second helical thread. The plurality of scallops comprising a scallop depth, the scallop depth substantially matching or matching the first and/or second helical thread depth. The plurality of scallops comprising a scallop shape, the scallop shape including a concave shape.

The third portion comprises a third portion shape. The third portion shape comprises a taper. The third portion further comprises at least two cutting flutes. The first portion of the shaft comprises a first portion length, the first portion length comprising at least 10% of the total shaft length. The first portion of the shaft comprises a first portion length, the first portion length comprising a range of 10% to 20% of total shaft length.

I/We claim:

1. A bone fixation screw comprising:
a shaft, the shaft comprising a shaft longitudinal axis, first end, a second end, total shaft length, a shaft diameter, a first portion, a second portion, and a third portion, the first portion adjacent to the first end, the second portion between the first portion and the third portion, the third portion adjacent to the second end, the first portion of the shaft comprises a first cross-sectional shape along the shaft longitudinal axis and a second cross-sectional shape along the shaft longitudinal axis, the second cross-sectional shape is perpendicular to the first cross-sectional shape, the first cross-sectional shape is different than the second cross-sectional shape;
a first helical thread, the first helical thread including a first thread depth and a first thread pitch; and
a second helical thread, the second helical thread including a second thread depth and a second thread pitch, the first helical thread and the second helical thread are disposed between the first end and the second end of the shaft and along the shaft longitudinal axis.

2. The bone fixation screw of claim 1, wherein the first helical thread and the second helical thread alternate along the shaft longitudinal axis.

3. The bone fixation screw of claim 1, wherein the first thread depth and the second thread depth are different.

4. The bone fixation crew of claim 1, wherein the first thread depth is larger than the second thread depth.

5. The bone fixation screw of claim 1, wherein the first helical thread or the second helical thread comprises a cutting thread.

6. The bone fixation screw of claim 1, wherein the first cross-sectional shape is circular and uniform along a first portion length, and the second cross-sectional is circular and non-uniform along the first portion length.

7. The bone fixation screw of claim 6, wherein the non-uniform along the first portion length comprises a taper.

8. The bone fixation screw of claim 1, wherein the first cross-sectional shape of the first portion comprises a first cross-sectional diameter and the second cross-sectional shape comprises a second-cross-sectional diameter, and the second portion of the shaft comprises a second portion shaft diameter, the first cross-sectional diameter substantially matches or matches the second portion shaft diameter.

9. The bone fixation screw of claim 1, wherein the first cross-sectional shape of the first portion comprises a first cross-sectional diameter and the second cross-sectional shape comprises a second-cross-sectional diameter, and the second portion of the shaft comprises a second portion shaft diameter, at least a portion of the second cross-sectional diameter is different the second portion shaft diameter.

10. The bone fixation screw of claim 1, wherein the first cross-sectional shape of the first portion comprises a first cross-sectional diameter and the second cross-sectional shape comprises a second-cross-sectional diameter, and the second portion of the shaft comprises a second portion shaft diameter, at least a portion of the second cross-sectional diameter is different than the second portion shaft diameter and the first cross-sectional diameter is the same or substantially the same as the second portion shaft diameter.

11. The bone fixation screw of claim 1, wherein the bone fixation screw further comprises a plurality of scallops, the plurality of scallops being sized and configured to receive a second bone fixation screw.

12. The bone fixation screw of claim 11, wherein the plurality of scallops is disposed onto a portion of the first helical thread or a portion of the second helical thread.

13. The bone fixation screw of claim 11, wherein the plurality of scallops is disposed onto a portion of the first helical thread and a portion of the second helical thread.

14. The bone fixation screw of claim 11, wherein the plurality of scallops comprising a scallop depth, the scallop depth substantially matching or matching the first and/or second helical thread depth.

15. The bone fixation screw of claim 11, wherein the plurality of scallops comprising a scallop shape, the scallop shape including a concave shape.

16. The bone fixation screw of claim 1, wherein the third portion comprises a third portion shape.

17. The bone fixation screw of claim 16, wherein the third portion shape comprises a taper.

18. The bone fixation screw of claim 1, wherein the third portion further comprises at least two cutting flutes.

19. The bone fixation screw of claim 1, wherein the first portion of the shaft comprises a first portion length, the first portion length comprising at least 10% of the total shaft length.

20. The bone fixation screw of claim 1, wherein the first portion of the shaft comprises a first portion length, the first portion length comprising a range of 10% to 20% of total shaft length.
